(19) United States
(12) Patent Application Publication
(10) Pub. No.: US 2019/0135877 A1
(43) Pub. Date: May 9, 2019

(54) RECOMBINANT KOMAGATAEIBACTER GENUS MICROORGANISM HAVING ENHANCED CELLULOSE PRODUCTIVITY, METHOD OF PRODUCING CELLULOSE USING THE SAME, AND METHOD OF PRODUCING THE MICROORGANISM

(71) Applicant: Samsung Electronics Co., Ltd., Suwon-si (KR)

(72) Inventors: Wooyong Shim, Suwon-si (KR); Soonchun Chung, Seoul (KR); Jinhwan Park, Suwon-si (KR)

(21) Appl. No.: 16/186,143

(22) Filed: Nov. 9, 2018

(30) Foreign Application Priority Data
Nov. 9, 2017 (KR) 10-2017-0148718

(51) Int. Cl. C07K 14/195; C12N 9/04; C12N 15/74; C12R 1/01; C12P 19/04; C12N 9/12; C12N 9/92

(52) U.S. Cl. CPC C07K 14/195; C12N 9/0006; C12N 15/74; C12R 1/01; C12Y 207/0109; C12N 9/1205; C12N 9/92; C12Y 101/01044; C12Y 503/01009; C12P 19/04

(57) ABSTRACT

A recombinant microorganism of the genus Komagataeibacter having enhanced cellulose productivity and yield, a method of producing cellulose using the recombinant microorganism, and a method of producing the recombinant microorganism are provided.

### CROSS-REFERENCE TO RELATED APPLICATION

This application claims the benefit of Korean Patent Application No. 10-2017-0148718, filed on Nov. 9, 2017, in the Korean Intellectual Property Office, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 52,524 Byte ASCII (Text) file named “740669_ST25.txt,” created on Nov. 9, 2018.

### BACKGROUND

### 1. Field

The present disclosure relates to a recombinant microorganism of the genus Komagataeibacter having enhanced cellulose productivity, a method of producing cellulose using the recombinant microorganism, and a method of producing the recombinant microorganism.

### 2. Description of the Related Art

Plant-based celluloses are abundant and inexpensive, and thus, are being examined for use in certain industries. However, lignocellulosic biomass needs to undergo complicated processing due to the presence of lignin, hemicelluloses, and other molecules, particularly when used in medical applications. Bacterial celluloses (BCs), on the other hand, are insoluble extracellular polysaccharides produced by bacteria such as that of the genus Acetobacter. Bacterial celluloses are present in the form of β-1,4 glucan as a primary structure, which then forms a network structure of several strands of fibrils. Bacterial celluloses are a highly pure form of cellulose with a fine nano-scale structure. Bacterial celluloses have excellent physico-chemical properties, including high mechanical tensile strength, purity, biodegradability, water-holding capacity, and high heat-resistance. Due to these properties, bacterial celluloses have been developed for applications in various industrial fields, including cosmetics, medicine, dietary fiber, vibration plates for sound systems, and functional films.

Microorganisms from the genera Acetobacter, Agrobacteria, Rhizobia, and Sarcina have been reported as bacterial cellulose-producing strains. Of these strains, Komagataeibacter xylinum (also called Gluconacetobacter xylinum) is known as a strain having excellent characteristics for producing cellulose. When Komagataeibacter xylinum is cultured under aerobic, static conditions, a 3-dimensional (3D) network structure of cellulose is formed as a thin film on a surface of a culture solution.

Therefore, there is a need to develop a recombinant microorganism of the genus Komagataeibacter having enhanced cellulose productivity.

### SUMMARY

Provided is a recombinant microorganism of the genus Komagataeibacter having enhanced cellulose productivity including a genetic modification that increases activity of 6-phosphogluconate dehydrogenase (GND).

Provided is a method of producing cellulose by using the recombinant microorganism by culturing the recombinant microorganism in a culture medium, thereby producing cellulose; and subsequently recovering the cellulose from the culture.

Provided is a method of producing the recombinant microorganism by introducing a gene that encodes 6-phosphogluconate dehydrogenase (GND) into a microorganism of the genus Komagataeibacter.

Additional aspects will be set forth in the description which follows and will be apparent from the description, or may be learned by practice of the presented embodiments.

### BRIEF DESCRIPTION OF THE DRAWING

These and/or other aspects will become apparent and more readily appreciated from the following description of the embodiments, taken in conjunction with the accompanying drawing in which:

FIG. 1 is a schematic diagram illustrating a structure of a DNA construct for introducing a GND or PGI gene into the genome of K. xylinus through homologous recombination.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the disclosure. As used herein, the term “and/or” includes any and all combinations of one or more of the associated listed items. Expressions such as “at least one of,” when preceding a list of elements, modify the entire list of elements and do not modify the individual elements of the list.

The terms “increase in activity”, or “increased activity” as used herein may refer to a detectable increase in activity of a cell, a protein, or an enzyme. The terms “increase in activity”, or “increased activity” as used herein may mean that a modified (for example, genetically engineered) cell, protein, or enzyme shows higher activity than a comparable cell, protein, or enzyme of the same type, like a cell, a protein, or an enzyme (for example, original or “wild-type” cell, protein, or enzyme) which does not have the genetic modification. The term “cell activity” as used herein may mean a cell activity specific to a particular protein or enzyme. For example, activity of a modified or engineered cell may be higher than activity of a non-engineered cell or parent cell of the same type, for example, a particular protein or enzyme of a wild-type cell by about 5% or more, about 10% or more, about 15% or more, about 20% or more, about 30% or more, about 50% or more, about 60% or more, about 70% or more, or about 100% or more. A cell including a protein or enzyme having increased enzymatic activity may be identified by any methods known in the art.

An increase in activity of an enzyme or polypeptide may be achieved by increasing expression or specific activity of the enzyme or polypeptide. The increase in expression may be achieved by introduction of a polynucleotide that encodes the enzyme or polypeptide into a cell. The increase in expression may also be achieved by an increase in the copy number of the polynucleotide encoding an enzyme or polypeptide, or by mutation of a regulatory region of the polynucleotide that increases expression. A microorganism into which the polynucleotide encoding the enzyme or polypeptide is introduced may or may not endogenously include the gene. The gene may be operably linked to a regulatory sequence that enables expression of the gene, for example, a promoter, a polyadenylation site, or a combination thereof. The polynucleotide that may be externally introduced or whose copy number may be increased may be endogenous or exogenous. An endogenous gene may refer to a gene that is intrinsically present in the genetic material of a microorganism. An exogenous gene may refer to a gene introduced into cells from outside. The introduced gene may be homologous or heterologous with respect to the host cell. The term “heterologous” refers to a gene that is “foreign,” or not “native” to the species.

The “copy number increase” of a gene as used herein may be due to the introduction of an exogenous gene or amplification of an endogenous gene, and may also include, for example, the introduction of an exogenous gene into a microorganism that did not previously include a copy of the gene. The introduction of a gene may be achieved via a vehicle such as a vector. The introduction of a gene may be transient introduction of the gene, lacking integration into the genome of the cell, or may be insertion of the gene into the genome. The introduction may be achieved, for example, through introduction of a vector into the cell, the vector including a polynucleotide encoding a target polypeptide, and then either the vector is replicated in the cell or the polynucleotide is integrated into the genome.

The introduction of a gene may be achieved by a known method, for example, transformation, transfection, or electroporation. The gene may be introduced via a vehicle or directly as it is. The term “vehicle” as used herein may also refer to a nucleic acid molecule that may deliver other nucleic acids linked thereto. The term “vehicle” as used herein may be used to refer to a vector, a nucleic acid construct, a cassette, or any other nucleic acid construct suitable for delivery of a gene. The vector may be, for example, a plasmid (e.g., plasmid expression vector), a viral vector (e.g., virus expression vector), or a combination thereof. Plasmids include circular double stranded DNA rings to which additional DNA may be linked. A viral vector may be, for example, a replication-defective retrovirus, an adenovirus, an adeno-associated virus, or a combination thereof.

The gene as used herein may be engineered or manipulated by any molecular biological method known in the art.

The term “parent cell” as used herein may refer to a cell that does not have a particular genetic modification as compared to a given modified microorganism, but is otherwise the same type of cell as the modified microorganism. Accordingly, the parent cell may be a cell that is used as a starting material for the production of a genetically engineered microorganism comprising a given modification (e.g., a modification that enhances activity of a protein, such as one of the genetic modifications described herein). The parent cell includes but is not limited to a “wild-type” cell. For example, in a microorganism in which a GND encoding gene is genetically modified to increase activity of the GND gene in a cell, the parent cell may be a microorganism that does not have the genetically modified GND encoding gene. The same comparison may apply to other types of genetic modification.

The term “gene” as used herein may refer to a nucleic acid fragment that encodes a particular protein, and may optionally include at least one regulatory sequence of a 5′-non-coding sequence and a 3′-non-coding sequence.

The term “sequence identity” of a polynucleotide sequence or polypeptide sequence as used herein refers to the degree of similarity between corresponding nucleotide or amino acid sequences measured after the sequences are optimally aligned. In some embodiments, a percentage of the sequence identity may be calculated by comparing two optimally aligned corresponding sequences in an entire comparable region, determining the number of locations where an amino acid residue or a nucleotide is identical in the two sequences to obtain the number of matched locations, dividing the number of the matched locations by the total number (that is, a range size) of all locations within a comparable range, and multiplying the result by 100 to obtain a percentage of the sequence identity. The percentage of the sequence identity may be determined by using known sequence comparison programs, examples of which include BLASTN (NCBI) and BLASTP (NCBI), CLC Main Workbench (CLC bio.), and MEGALIGN™ (DNASTAR Inc).

In identifying polypeptides or polynucleotides of different species that may have an identical or similar function or activity, varying levels of sequence identity may be used. For example, the sequence identity may be about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, about 99% or more, or 100%.

The term “genetic modification” as used herein may refer to an artificial change in the composition or structure of the genetic material of a cell.

According to one aspect of the present invention, a recombinant microorganism of the genus Komagataeibacter having enhanced cellulose productivity comprises a genetic modification that increases activity of 6-phosphogluconate dehydrogenase (GND).

The microorganism may further include a genetic modification that increases activity of phosphoglucose isomerase (PGI).

The genetic modification that increases activity of 6-phosphogluconate dehydrogenase (GND) and the genetic modification that increases activity of phosphoglucose isomerase (PGI) may respectively increase the expression of a gene that encodes the GND and a gene that encodes the PGI. The genetic modifications may also increase the copy number of the gene that encodes the GND and/or the gene that encodes the PGI. For instance, the genetic modifications may include introducing one or more exogenous polynucleotides encoding the GND and/or PGI. The genetic modifications may also be modifications of the expression regulatory sequences of the genes that encode the GND and/or the PGI.

The GND is an enzyme involved in a pentose phosphate pathway. The GND may catalyze decarboxylating reduction of 6-phosphogluconate into ribulose 5-phosphate in the presence of nicotinamide adenine dinucleotide phosphate (NADP). The GND may belong to EC 1.1.1.44. The GND may be a polypeptide having a sequence identity of about 85% or greater, about 90% or greater, about 95% or greater, or about 100% with the amino acid sequence of SEQ ID NO: 1.

The PGI may belong to EC 5.3.1.9. The PGI may catalyze interconversion between fructose-6-phosphate and glucose-6-phosphate. The PGI may be a polypeptide having a sequence identity of about 85% or greater, 90% or greater, 95% or greater, or about 100% with the amino acid sequence of SEQ ID NO: 3 or SEQ ID NO: 5.

The microorganism may further include a genetic modification that increases activity of phosphofructose kinase (PFK).

The genetic modification may increase expression of a gene that encodes the PFK. The genetic modification may increase the copy number of a gene that encodes the PFK or modify an expression regulatory sequence of a gene that encodes the PFK. The copy number increase may be achieved by introduction of one or more exogenous polynucleotides encoding the PFK.

The PFK is a protein that phosphorylates fructose-6-phosphate into fructose-1,6-bisphosphate in glycolysis. The PFK may be exogenous or endogenous. The PFK may be PFK1 (referred to also as “PFKA”). The PFK or PFK1 may belong to EC 2.7.1.11. The PFK1 may be of a bacterial origin. The PFK1 may be derived, for instance, from the genus Escherichia, the genus Bacillus, the genus Mycobacterium, the genus Zymomonas, or the genus Vibrio. The PFK1 may be derived from E. coli, for example, E. coli MG1655.

The PFK1 may catalyze conversion of ATP and fructose-6-phosphate to ADP and fructose-1,6-bisphosphate. The PFK1 may be allosterically activated by ADP and diphosphonucleoside and may be allosterically inhibited by phosphoenolpyruvate. The PFK1 may be a polypeptide having a sequence identity of about 85% or greater, about 90% or greater, about 95% or greater, or about 100% with an amino acid sequence of SEQ ID NO: 20.

The genetic modification may be achieved by introducing at least one of the gene that encodes the GND and the gene that encodes the PGI, for example, via a vehicle such as a vector. The introduced at least one of the gene that encodes the GND and the gene that encodes the PGI may or may not be integrated into the genome of the microorganism. A plurality of the gene encoding the GND or the gene encoding the PGI may be introduced, for example, 2 or more, 5 or more, 10 or more, 30 or more, 50 or more, 100 or more, or 1,000 or more.

The genetic modification may be achieved by introducing a gene that encodes the PFK, for example, via a vehicle such as a vector. The gene that encodes the PFK may or may not be chromosome integrated into the genome of the microorganism. The number of the introduced genes that encode the PFK may be plural, for example, 2 or more, 5 or more, 10 or more, 30 or more, 50 or more, 100 or more, or 1,000 or more.

The recombinant microorganism may have enhanced bacterial cellulose productivity, and may belong to the genus Komagataeibacter, the genus Acetobacter, or the genus Gluconacetobacter. The microorganism may be K. xylinus (referred to also as “G. xylinus”), K. rhaeticus, K. swingsii, K. kombuchae, K. nataicola, or K. sucrofermentans.

According to another aspect of the present invention, a method of producing cellulose comprises: culturing a recombinant microorganism of the genus Komagataeibacter having enhanced cellulose productivity in a culture medium to thereby produce cellulose, the microorganism including at least one of a genetic modification that increases activity of 6-phosphogluconate dehydrogenase (GND) and a genetic modification that increases activity of phosphoglucose isomerase (PGI); and recovering the cellulose from the culture.

The recombinant microorganism may be the same recombinant microorganism provided herein.

The culturing may be performed in a culture medium including a carbon source, for example, glucose. The culture medium used in the culturing of the microorganism may be any general culture medium appropriate for growth of a host cell, such as a minimal medium or a complex medium including an appropriate supplement. An appropriate medium may be commercially purchased or may be prepared using a known preparation method.

The culture medium may be a medium containing selected ingredients satisfying the specific requirements of a microorganism. The culture medium may be a medium including an ingredient selected from a carbon source, a nitrogen source, a salt, a trace element, or a combination thereof.

The culturing conditions may be appropriately controlled for the production of a selected product, for example, cellulose. The culturing may be performed under aerobic conditions for cell proliferation. The culturing may be performed by spinner culture or by static culture without shaking. A concentration of the microorganism may be such that a density of the microorganism gives enough space so as not to disturb production of cellulose.

The term “culturing condition” as used herein refers to a condition for culturing the microorganism. The culturing condition may be, for example, a carbon source, a nitrogen source, or oxygen used by the microorganism. The carbon source that is usable by the microorganism may include a monosaccharide, a disaccharide, or a polysaccharide. The carbon source may be an assimilable carbon source for any microorganism. For example, the carbon source may be glucose, fructose, mannose, or galactose. The nitrogen source may be an organic nitrogen compound or an inorganic nitrogen compound. The nitrogen source may be, for example, an amino acid, an amide, an amine, a nitrate, or an ammonium salt. The oxygen condition for culturing the microorganism may be an aerobic condition at a normal partial pressure of oxygen, or an atmospheric low-oxygen condition including about 0.1% to about 10% oxygen in air. A metabolic pathway of the microorganism may vary in accordance with the carbon source and nitrogen source that are practically available.

The culture medium may include ethanol or cellulose. An amount of the ethanol may be about 0.1 to about 5 (v/v)%, about 0.3 to about 2.5 (v/v)%, about 0.3 to about 2.0 (v/v)%, about 0.3 to about 1.5 (v/v)%, about 0.3 to about 1.25 (v/v)%, about 0.3 to about 1.0 (v/v)%, about 0.3 to about 0.7 (v/v)%, or about 0.5 to about 3.0 (v/v)% based on the total volume of the culture medium. An amount of the cellulose may be about 0.5 to about 5 (w/v)%, about 0.5 to about 2.5 (w/v)%, about 0.5 to about 1.5 (w/v)%, or about 0.7 to about 1.25 (w/v)% based on the total volume of the culture medium. The cellulose may be a carboxylated cellulose. The cellulose may be a carboxyl methylcellulose (CMC). For example, the CMC may be sodium carboxyl methylcellulose.

The method may include separating the cellulose from the culture. The separating may be, for example, recovering a cellulose pellicle formed on the surface of the culture medium. The cellulose pellicle may be recovered by being physically removed, or by removing the culture medium. The separating may include recovering the cellulose pellicle intact without damaging the shape of the cellulose pellicle.

According to another aspect of the present invention, a method of producing a microorganism having enhanced cellulose productivity includes introducing at least one of a gene that encodes 6-phosphogluconate dehydrogenase (GND) and a gene that encodes phosphoglucose isomerase (PGI) into a microorganism of the genus Komagataeibacter. The introduction of the gene that encodes the GND and/or the PGI may be an introduction of a vehicle comprising the gene into the microorganism. In the method according to one or more embodiments, a genetic modification may include any of amplifying the gene, manipulating a regulatory sequence of the gene, and/or manipulating the sequence of the gene itself. The manipulating may include any of insertion, substitution, conversion, and/or addition of one or more nucleotides.

In some embodiments, the method may further include introducing a gene that encodes PFK into the microorganism.

The recombinant microorganism of the genus Komagataeibacter having enhanced cellulose productivity, according to any of the embodiments, may be used to produce cellulose with high efficiency.

The method of producing cellulose, according to any of the embodiments, may be used to efficiently produce cellulose.

The method of producing the recombinant microorganism having enhanced cellulose productivity, according to any of the embodiments, may be used to efficiently produce the recombinant microorganism having enhanced cellulose productivity.

One or more embodiments of the present invention will now be described in detail with reference to the following examples. However, these examples are only for illustrative purposes and are not intended to limit the scope of the one or more embodiments of the present invention.

### EXAMPLE 1

### Construction of K. xylinus Comprising Over-Expressed Heterologous 6-phosphogluconate Dehydrogenase (GND) Gene or phosphoglucose Isomerase (PGI) Gene, and Production of Cellulose

In the present example, a foreign GND gene or PGI gene was introduced into a genome of Komagataeibacter xylinus KCCM 41431 (available from the Korean Culture Center of Microorganisms (KCCM)), and the gene-introduced microorganism was cultured to allow the microorganism to consume glucose and produce cellulose, in order to determine an effect of the introduction of the gene on cellulose productivity.

1. Construction of Vector for GND and PGI Overexpression

PCR was carried out using the genomic DNA of Escherichia coli (E. coli) and Corynebacterium glutamicum as a template and primer sets (SEQ ID NOs: 12 and 13; SEQ ID NOs: 14 and 15; and SEQ ID NOs: 16 and 17) to amplify open reading frames (ORFs) of PGI genes of E. coli and C. glutamicum (SEQ ID NO: 4 and SEQ ID NO: 6) and a GND gene of C. glutamicum (SEQ ID NO: 2), which were then extracted by gel extraction. The resulting gene fragments were cloned into a pJET-EX vector (SEQ ID NO: 7) using an IN-FUSION® GD Cloning kit (available Takara, Japan) to construct expression vectors each including a gene construct of tac promoter-gene ORF-rrnBT terminator, that is, pJET_ecPGI, pJET_cgPGI, pJET_ecGND, and pJET_cgGND. The pJET-EX vector was a pJET1.2 vector (available from ThermoScientific) with the tac promoter and the rrnBT terminator inserted thereinto. The tac promoter and rrnBT terminator in the gene construct were verified to be permanently operable in cells of the genus Komagateibacter. The pJET vector is a cloning vector that is not replicable in both E. coli and X. xylinus.

2. Construction of Cassette Vector for Insertion at sacB Gene Locus

The levansucrase (sacB) gene locus in the chromosome of K. xylinus KCCM 41431 was chosen as an insertion site for the PGI gene and GND gene expression constructs. Vectors were constructed for generating a control strain for determining an effect of introduction of the PGI and GND genes, wherein the control strain was a strain with only a kanamycin marker inserted at the sacB gene site. These vectors included a homologous arm sequence in the 5′-upstream region and the 3′-downstream region of the sacB gene for insertion by double crossover homologous recombination.

In particular, PCR was carried out using the genomic DNA of K. xylinus KCCM 41431 as a template, a sacB_left forward and reverse primer set (SEQ ID NOs: 8 and 9), and a sacB_right forward and reverse primer set (SEQ ID NOs: 10 and 11) to obtain PCR products of 0.8 kb and 0.7 kb, respectively, which were then inserted at XbaI and EcoRI restriction enzyme loci of the pMKO vector (SEQ ID NO: 39) using an IN-FUSION® GD Cloning kit (available from Takara, Japan) to construct a pMKO_(del)sacB vector. The pMKO-(del)sacB vector had a kanamycin resistance gene expression construct, i.e., a gap promoter-kanamycin resistance gene-rnnBT terminator, as a selection marker for identifying whether or not the chromosomal insertion occurred.

3. Construction of Vector for Insertion of PGI Gene and GND Gene Expression Constructs at sacB Gene Site

To insert a gene construct for expression of GND and PGI genes into the constructed pMKO_(del)sacB vector, i.e., a Ptac promoter-gene ORF-rrnBT terminator, PCR was carried out using pJET_ecPGI, pJET_cgPGI, and pJET_cgGND vectors as templates and a pJET_geneset forward and reverse primer set (SEQ ID NOs: 41 and 42) to obtain amplified products of the PGI gene expression construct and the GND gene expression construct. These amplified products were then cloned at XbaI restriction enzyme sites of the pMKO_(del)sacB vector using an IN-FUSION® GD Cloning kit (available from Takara, Japan) to construct pMKO-(del)sacB_ecPGI, pMKO-(del) sacB_cgPGI, and pMKO-(del)sacB_cgGND vectors.

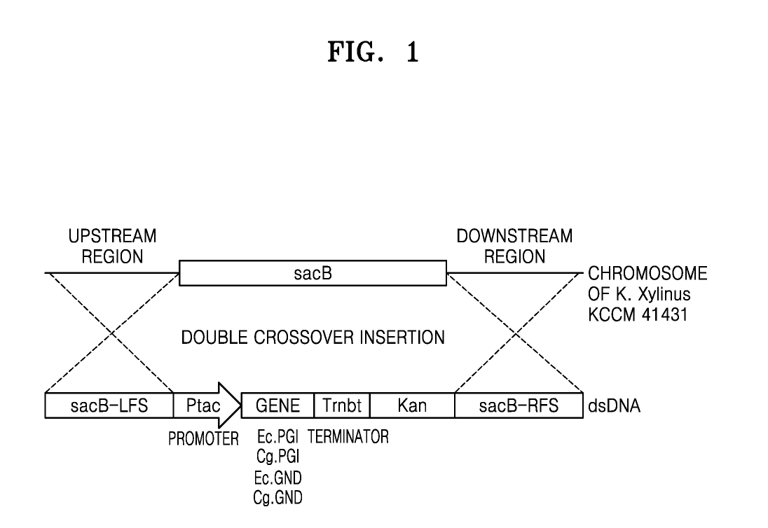

FIG. 1 is a schematic diagram illustrating a structure of a DNA construct for introducing a GND or PGI gene into a genome of K. xylinus, a genome sequence, and homologous recombination.

4. Chromosomal Insertion of GND Gene and PGI Gene Constructs

To introduce GND and PGI gene expression cassettes into the K. xylinus strain, PCR was carried out using pMKO-(del)sacB_ecPGI, pMKO-(del) sacB_cgPGI, and pMKO-(del)sacB_cgGND vectors as templates and a primer set of SEQ ID NO: 18 and SEQ ID NO: 19 to amplify the gene insertion cassettes. The amplified gene insertion cassettes were then introduced into the K. xylinus strain by transformation as follows.

The K. xylinus KCCM 41431 strain was then spread over a plate smeared with a 2%-glucose added HS medium (containing 0.5% of peptone, 0.5% of yeast extract, 0.27% of Na₂HPO₄, 0.15% of citric acid, 2% of glucose, and 1.5% of bacto-agar), and cultured at about 30° C. for 3 days. This cultured strain was transferred to a 50-mL falcon tube using sterilized water and then vortexed for about 2 minutes. After 0.1 (v/v)% of cellulase (cellulase from Trichoderma reesei ATCC 26921, available from Sigma) was added thereto and reacted at about 30° C. at about 160 rpm for about 2 hours, the reaction product was washed with a 1-mM HEPES buffer and then with 15 (w/v)% of glycerol three times, and then re-suspended in 1 mL of 10 (w/v)% glycerol to construct competent cells.

After 100 ul of the constructed competent cells was transferred to a 2-mm electro-cuvette and 3 μg of the constructed DNA cassette was added thereto, the DNA cassette was introduced into the cells by electroporation (2.4 kV, 200Ω, 25 μF). Then, 1 mL of a HS medium was added thereto, re-suspended, and transferred to a 14-mL round-bottomed tube, and cultured at about 30° C. at about 160 rpm for about 2 hours. This cultured product was spread over a plate smeared with a HS medium containing 2(w/w)% of glucose, 1 (v/v)% of ethanol and 5 ug/mL of kanamycin added thereto, and then cultured at about 30° C. for about 5 days to induce homologous recombination.

5. Production of Cellulose

The K. xylinus strain obtained by introducing the DNA expression construct at the sacB locus of the genomic DNA of the K. xylinus KCCM 41431 strain was streaked on a plate smeared with an HS medium containing 2 (w/w)% glucose, 1 (v/v)% of ethanol, and 5 ug/mL of kanamycin, and then cultured at about 30° C. for about 5 days. This cultured strain was inoculated into 50 mL of an HS medium containing 4% of glucose and 1% of ethanol added thereto and then cultured at about 30° C. at about 230 rpm for about 5 days. The produced cellulose was then washed at about 60° C. with 0.1N NaOH and distilled water, freeze-dried to remove H₂O, and weighed. Glucose and gluconate contents were analyzed by high-performance liquid chromatography (HPLC). Table 2 shows the results of component analysis of each culture, and in particular, the produced amount and yield of cellulose in each K. xylinus strain into which the exogenous PGI or GND gene was introduced.

TABLE 2

| | CNF (g/L) | Yield of CNF (g/g) (%) |
|---|---|---|
| WT | 1.5 | 5.0 |
| WTΔsacB | 1.4 | 4.8 |
| ΔsacB Ptac::Ec. PGI | 3.1 | 8.7 |
| ΔsacB Ptac::Cg. PGI | 2.9 | 7.9 |
| ΔsacB Ptac::Cg. GND | 2.2 | 6.8 |

Referring to Table 2, the K. xylinus strains into which the PGI gene or GND gene was introduced were found to produce increased amounts of cellulose with higher yields as compared to the strains lacking the foreign PGI gene or GND gene.

### EXAMPLE 2

### Construction of K. xylinus Including PFK Gene and GND Gene or PGI Gene, and Production of Cellulose

The same processes as described above in Example 1 were performed, except that K. xylinus in which the PFK gene introduced into the genome thereof was used as a starting strain, and the GND gene or PGI gene was introduced into the starting strain. The processes in Example 2 are the same as those of Example 1, unless stated otherwise.

1. Construction of Vector for pfkA Overexpression

The phosphofructose kinase (pfk) gene was introduced into K. xylinus by homologous recombination as follows.

PCR was carried out using the pTSa-EX1 vector (SEQ ID NO: 22) as a template, a primer set of SEQ ID NO: 23 and SEQ ID NO: 24, and a primer set of SEQ ID NO: 25 and SEQ ID NO: 26 to obtain an amplified product. This amplified product was cloned at the BamHI and SalI restriction enzyme loci of the pTSa-EX1 vector using an IN-FUSION® GD Cloning kit (available from Takara, Japan) to construct a pTSa-EX11 vector. The pTSa-EX1 vector is a shuttle vector that is replicable in both E. coli and X. xylinus.

To introduce the pfkA gene by homologous recombination, an open reading frame (ORF) (SEQ ID NO: 21) of the pfkA gene was obtained by PCR using the genomic DNA of the E. coli K12 MG1655 as a template and a primer set of SEQ ID NO: 27 and SEQ ID NO: 28. The pfkA gene fragment was cloned at the BamHI and SalI restriction enzyme loci of the pTSa-EX11 vector using an IN-FUSION® GD Cloning kit (available from Takara, Japan), thereby constructing a pTSa-Ec.pfkA vector for overexpressing the pfkA gene.

2. Construction of Vector for E. coli pfkA Gene Insertion

PCR was performed using the pTSa-Ec.pfkA vector as a template and a primer set of SEQ ID NO: 29 and SEQ ID NO: 30 to amplify the tetA gene. This PCR product was cloned at the EcoRI restriction enzyme locus of the pMSK+ vector (Genbank Accession No. KJ922019) using an IN-FUSION® GD Cloning kit (available from Takara, Japan) to construct a pTSK+ vector.

Then, PCR was carried out using the genomic DNA of K. xylinus strain as a template and primer sets (SEQ ID NO: 31/32, SEQ ID NO: 33/34, and SEQ ID NO: 35/36) to amplify a homologous region of the pfkA gene insertion locus. This PCR product was cloned at the EcoRI restriction enzyme locus of the pTSK+ vector using an IN-FUSION® GD Cloning kit (available from Takara, Japan) to construct a pTSK-(del)2760 vector.

PCR was carried out using the pTSa-Ec.pfkA vector as a template and a primer set of SEQ ID NO: 37 and SEQ ID NO: 38 to amplify the Ptac::Ec.pfkA gene. This PCR product was cloned at the EcoRI restriction enzyme locus of the pTSK-(del)2760 vector using an IN-FUSION® GD Cloning kit (available from Takara, Japan) to construct a pTSK-(del)2760-Ec.pfkA vector.

3. Introduction of Phosphofructose Kinase (pfkA) Gene

To introduce E. coli pfkA gene, i.e., a nucleotide sequence of SEQ ID NO: 21, into K. xylinus, PCR was carried out using the pTSK-(del)2760-Ec.pfkA vector as a template and a primer set of SEQ ID NO: 31 and SEQ ID NO: 36 to amplify a cassette for Ptac::Ec.pfkA gene insertion. This cassette for Ptac::Ec.pfkA gene insertion was then introduced into K. xylinus strain by transformation as follows.

The K. xylinus strain was smeared on a 2%-glucose added HS medium (containing 0.5% of peptone, 0.5% of yeast extract, 0.27% of Na₂HPO₄, 0.15% of citric acid, 2% of glucose, and 1.5% of bacto-agar) and then cultured at about 30° C. for about 3 days. This cultured strain was inoculated into 5 mL of a HS medium to which 0.2 (v/v)% of cellulase (cellulase from Trichoderma reesei ATCC 26921, available from Sigma) was added, and then cultured at about 30° C. for about 2 days. This cultured cell suspension was inoculated into 100 mL of the HS medium to which 0.2 (v/v)% of cellulose was added, until the cell density (OD₆₀₀) reached 0.04, and then cultured at about 30° C. to a cell density (OD₆₀₀) of about 0.4 to about 0.7. The cultured strain was washed with 1 mM of a HEPES buffer and then with 15(w/v)% of glycerol three times, and then re-suspended in 1 mL of 15(w/v)% of glycerol to construct competent cells.

After 100 ul of the constructed competent cells was transferred to a 2-mm electro-cuvette, and 3 ug of the Ptac::Ec.pfkA cassette constructed above in Section 2 was added thereto, the vector including the cassette was introduced into the competent cells by electroporation (2.4 kV, 200Ω, 25 μF). The vector-introduced cells were re-suspended in 1 mL of the HS medium containing 2(w/v)% of glucose and 0.1 (v/v)% of cellulose, and the re-suspended cells were transferred to a 14-mL of a round-bottomed tube, and then cultured at about 30° C. at about 160 rpm for about 16 hours. The cultured cells were smeared on a HS medium containing 2(w/v)% of glucose, 1(v/v)% of ethanol, and 5 ug/mL of tetracycline added thereto, and cultured at about 30° C. for about 4 days to select a strain having tetracycline resistance, thereby constructing a pfk gene-overexpressed strain (hereinafter, referred to also as “SK3 strain”).

4. Production of Cellulose

The pfk gene-overexpressed SK3 strain was used, and the C. glutamicum-derived PGI gene or GND gene was inserted into the genome of the SK3 strain in a manner according to Example 1. Each strain was then cultured as described above in Example 1 to recover cellulose. Table 3 shows a produced amount and yield of cellulose in each K. xylinus strain into which the exogenous PGI or GND, and PFK, were introduced.

TABLE 3

| | CNF (g/L) | Yield of CNF (g/g) (%) |
|---|---|---|
| SK3 | 3.5 | 10.5 |
| SK3ΔsacB | 3.3 | 10.3 |
| SK3ΔsacB Ptac::Cg. PGI | 4.3 | 11.3 |
| SK3ΔsacB Ptac::Cg. GND | 3.8 | 11.3 |

Referring to Table 3, the pfk gene-overexpressed strains into which the PGI or GND gene was further introduced were found to produce increased amounts of cellulose compared to the strains lacking the foreign PGI or GND gene.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant Komagataeibacter microorganism having enhanced cellulose productivity, the microorganism comprising a genetic modification that increases activity of 6-phosphogluconate dehydrogenase (GND).

2. The recombinant microorganism of claim 1, wherein the genetic modification increases expression of a gene that encodes GND.

3. The recombinant microorganism of claim 1, wherein the genetic modification is an increase in the copy number of a gene that encodes GND, or modification of an expression regulatory sequence of a gene that encodes GND.

4. The recombinant microorganism of claim 1, wherein GND belongs to EC 1.1.1.44.

5. The recombinant microorganism of claim 1, wherein GND has about 85% or more sequence identity with SEQ ID NO: 1.

6. The recombinant microorganism of claim 1, further comprising at least one of a genetic modification that increases activity of phosphofructose kinase (PFK) and a genetic modification that increases activity of phosphoglucose isomerase (PGI).

7. The recombinant microorganism of claim 1, further comprising at least one of a genetic modification that increases expression of a gene that encodes PFK and a genetic modification that increases expression of a gene that encodes PGI.

8. The recombinant microorganism of claim 1, further comprising at least one of a genetic modification that increases a copy number of a gene that encodes PFK, a genetic modification that increases a copy number of a gene that encodes PGI, a modification of an expression regulatory sequence of a gene that encodes PFK, and a modification of an expression regulatory sequence of a gene that encodes PGI.

9. The recombinant microorganism of claim 6, wherein PFK and PGI belong to EC 2.7.1.11 and EC 5.3.1.9, respectively.

10. The recombinant microorganism of claim 6, wherein PFK has about 85% or more sequence identity with SEQ ID NO: 20, and PGI has about 85% or more sequence identity with SEQ ID NO: 3 or SEQ ID NO: 5.

11. The recombinant microorganism of claim 1, wherein the recombinant microorganism is Komagataeibacter xylinus.

12. A method of producing cellulose, the method comprising:
culturing the recombinant microorganism of claim 1 in a culture medium to produce cellulose; and
recovering the cellulose from the culture.

13. The method of claim 12, wherein the genetic modification increases expression of a gene that encodes GND.

14. The method of claim 12, wherein the genetic modification is an increase in a copy number of a gene that encodes GND, or modification of an expression regulatory sequence of a gene that encodes GND.

15. The method of claim 12, wherein the recombinant microorganism further comprises at least one of a genetic modification that increases activity of phosphofructose kinase (PFK) and a genetic modification that increases activity of phosphoglucose isomerase (PGI).

16. The method of claim 12, wherein further comprising at least one of a genetic modification that increases expression of a gene that encodes PFK and a genetic modification that increases expression of a gene that encodes PGI.

17. The method of claim 12, further comprising at least one of a genetic modification that increases a copy number of a gene that encodes PFK, a genetic modification that increases a copy number of a gene that encodes PGI, a modification of an expression regulatory sequence of a gene that encodes PFK, and a modification of an expression regulatory sequence of a gene that encodes PGI.

18. The method of claim 12, wherein the recombinant microorganism is Komagataeibacter xylinus.

19. The method of claim 12, wherein the culture medium comprises about 0.5 w/v% to about 5.0 w/v% of CMC, about 0.1 v/v% to about 5.0 v/v% of ethanol, or about 0.5 w/v% to about 5.0 w/v% of CMC and about 0.1 v/v% to about 5.0 v/v% of ethanol.

20. A method of producing a microorganism of claim 1 having enhanced cellulose productivity, the method comprising introducing a gene that encodes 6-phosphogluconate dehydrogenase (GND) into a Komagataeibacter microorganism.


SEQUENCE LISTING (42 sequences)

SEQ ID NO: 1 - protein, 484 aa, Corynebacterium glutamicum
MTNGDNLAQIGVVGLAVMGSNLARNFARNGNTVAVYNRSTDKTDKLIADHGSEGNFIPSATVEEFVASLEKPRRAIIMVQAGNATDAVINQLADAMDEGDIIIDGGNALYTDTIRREKEISARGLHFVGAGISGGEEGALNGPSIMPGGPAKSYESLGPLLESIAANVDGTPCVTHIGPDGAGHFVKMVHNGIEYADMQVIGEAYHLLRYAAGMQPAEIAEVFKEWNAGDLDSYLIEITAEVLSQVDAETGKPLIDVIVDAAGQKGTGRWTVKAALDLGIATTGIGEAVFARALSGATSQRAAAQGNLPAGVLTDLEALGVDKAQFVEDVRRALYASKLVAYAQGFDEIKAGSDENNWDVDPRDLATIWRGGCIIRAKFLNRIVEAYDANAELESLLLDPYFKSELGDLIDSWRRVIVTATQLGLPIPVFASSLSYYDSLRAERLPAALIQGQRDFFGAHTYKRIDKDGSFHTEWSGDRSEVEA

SEQ ID NO: 2 - DNA, 1455 bp, Corynebacterium glutamicum
atgactaatggagataatctcgcacagatcggcgttgtaggcctagcagtaatgggctcaaacctcgcccgcaacttcgcccgcaacggcaacactgtcgctgtctacaaccgcagcactgacaaaaccgacaagctcatcgccgatcacggctccgaaggcaacttcatcccttctgcaaccgtcgaagagttcgtagcatccctggaaaagccacgccgcgccatcatcatggttcaggctggtaacgccaccgacgcagtcatcaaccagctggcagatgccatggacgaaggcgacatcatcatcgacggcggcaacgccctctacaccgacaccattcgtcgcgagaaggaaatctccgcacgcggtctccacttcgtcggtgctggtatctccggcggcgaagaaggcgcactcaacggcccatccatcatgcctggtggcccagcaaagtcctacgagtccctcggaccactgcttgagtccatcgctgccaacgttgacggcaccccatgtgtcacccacatcggcccagacggcgccggccacttcgtcaagatggtccacaacggcatcgagtacgccgacatgcaggtcatcggcgaggcataccaccttctccgctacgcagcaggcatgcagccagctgaaatcgctgaggttttcaaggaatggaacgcaggcgacctggattcctacctcatcgaaatcaccgcagaggttctctcccaggtggatgctgaaaccggcaagccactaatcgacgtcatcgttgacgctgcaggtcagaagggcaccggacgttggaccgtcaaggctgctcttgatctgggtattgctaccaccggcatcggcgaagctgttttcgcacgtgcactctccggcgcaaccagccagcgcgctgcagcacagggcaacctacctgcaggtgtcctcaccgatctggaagcacttggcgtggacaaggcacagttcgtcgaagacgttcgccgtgcactgtacgcatccaagcttgttgcttacgcacagggcttcgacgagatcaaggctggctccgacgagaacaactgggacgttgaccctcgcgacctcgctaccatctggcgcggcggctgcatcattcgcgctaagttcctcaaccgcatcgtcgaagcatacgatgcaaacgctgaacttgagtccctgctgctcgatccttacttcaagagcgagctcggcgacctcatcgattcatggcgtcgcgtgattgtcaccgccacccagcttggcctgccaatcccagtgttcgcttcctccctgtcctactacgacagcctgcgtgcagagcgtctgccagcagccctgatccaaggacagcgcgacttcttcggtgcgcacacctacaagcgcatcgacaaggatggctccttccacaccgagtggtccggcgaccgctccgaggttgaagcttaa

SEQ ID NO: 3 - protein, 549 aa, Escherichia coli
MKNINPTQTAAWQALQKHFDEMKDVTIADLFAKDGDRFSKFSATFDDQMLVDYSKNRITEETLAKLQDLAKECDLAGAIKSMFSGEKINRTENRAVLHVALRNRSNTPILVDGKDVMPEVNAVLEKMKTFSEAIISGEWKGYTGKAITDVVNIGIGGSDLGPYMVTEALRPYKNHLNMHFVSNVDGTHIAEVLKKVNPETTLFLVASKTFTTQETMTNAHSARDWFLKAAGDEKHVAKHFAALSTNAKAVGEFGIDTANMFEFWDWVGGRYSLWSAIGLSIVLSIGFDNFVELLSGAHAMDKHFSTTPAEKNLPVLLALIGIWYNNFFGAETEAILPYDQYMHRFAAYFQQGNMESNGKYVDRNGNVVDYQTGPIIWGEPGTNGQHAFYQLIHQGTKMVPCDFIAPAITHNPLSDHHQKLLSNFFAQTEALAFGKSREVVEQEYRDQGKDPATLDYVVPFKVFEGNRPTNSILLREITPFSLGALIALYEHKIFTQGVILNIFTFDQWGVELGKQLANRILPELKDDKEISSHDSSTNGLINRYKAWRG

SEQ ID NO: 4 - DNA, 1650 bp, Escherichia coli
atgaaaaacatcaatccaacgcagaccgctgcctggcaggcactacagaaacacttcgatgaaatgaaagacgttacgatcgccgatctttttgctaaagacggcgatcgtttttctaagttctccgcaaccttcgacgatcagatgctggtggattactccaaaaaccgcatcactgaagagacgctggcgaaattacaggatctggcgaaagagtgcgatctggcgggcgcgattaagtcgatgttctctggcgagaagatcaaccgcactgaaaaccgcgccgtgctgcacgtagcgctgcgtaaccgtagcaataccccgattttggttgatggcaaagacgtaatgccggaagtcaacgcggtgctggagaagatgaaaaccttctcagaagcgattatttccggtgagtggaaaggttataccggcaaagcaatcactgacgtagtgaacatcgggatcggcggttctgacctcggcccatacatggtgaccgaagctctgcgtccgtacaaaaaccacctgaacatgcactttgtttctaacgtcgatgggactcacatcgcggaagtgctgaaaaaagtaaacccggaaaccacgctgttcttggtagcatctaaaaccttcaccactcaggaaactatgaccaacgcccatagcgcgcgtgactggttcctgaaagcggcaggtgatgaaaaacacgttgcaaaacactttgcggcgctttccaccaatgccaaagccgttggcgagtttggtattgatactgccaacatgttcgagttctgggactgggttggcggccgttactctttgtggtcagcgattggcctgtcgattgttctctccatcggctttgataacttcgttgaactgctttccggcgcacacgcgatggacaagcatttctccaccacgcctgccgagaaaaacctgcctgtactgctggcgctgattggcatctggtacaacaatttctttggtgcggaaactgaagcgattctgccgtatgaccagtatatgcaccgtttcgcggcgtacttccagcagggcaatatggagtccaacggtaagtatgttgaccgtaacggtaacgttgtggattaccagactggcccgattatctggggtgaaccaggcactaacggtcagcacgcgttctaccagctgatccaccagggaaccaaaatggtaccgtgcgatttcatcgctccggctatcacccataacccgctctctgatcatcaccagaaactgctgtctaacttcttcgcccagaccgaagcgctggcgtttggtaaatcccgcgaagtggttgagcaggaatatcgtgatcagggtaaagatccggcaacgcttgactacgtggtgccgttcaaagtattcgaaggtaaccgcccgaccaactccatcctgctgcgtgaaatcactccgttcagcctgggtgcgttgattgcgctgtatgagcacaaaatctttactcagggcgtgatcctgaacatcttcaccttcgaccagtggggcgtggaactgggtaaacagctggcgaaccgtattctgccagagctgaaagatgataaagaaatcagcagccacgatagctcgaccaatggtctgattaaccgctataaagcgtggcgcggttaa

SEQ ID NO: 5 - protein, 540 aa, Corynebacterium glutamicum
MADISTTQVWQDLTDHYSNFQATTLRELFKEENRAEKYTFSAAGLHVDLSKNLLDDATLTKLLALTEESGLRERIDAMFAGEHLNNTEDRAVLHTALRLPAEADLSVDGQDVAADVHEVLGRMRDFATALRSGNWLGHTGHTIKKIVNIGIGGSDLGPAMATKALRAYATAGISAEFVSNVDPADLVSVLEDLDAESTLFVIASKTFTTQETLSNARAARAWLVEKLGEEAVAKHFVAVSTNAEKVAEFGIDTDNMFGFWDWVGGRYSVDSAVGLSLMAVIGPRDFMRFLGGFHAMDEHFRTTKFEENVPILMALLGVWYSDFYGAETHAVLPYSEDLSRFAAYLQQLTMESNGKSVHRDGSPVSTGTGEIYWGEPGTNGQHAFFQLIHQGTRLVPADFIGFARPKQDLPAGERTMHDLLMSNFFAQTKVLAFGKNAEEIAAEGVAPELVNHKVMPGNRPTTTILAEELTPSILGALIALYEHIVMVQGVIWDINSFDQWGVELGKQQANDLAPAVSGEEDVDSGDSSTDSLIKWYRANR

SEQ ID NO: 6 - DNA, 1623 bp, Corynebacterium glutamicum
atggcggacatttcgaccacccaggtttggcaagacctgaccgatcattactcaaacttccaggcaaccactctgcgtgaacttttcaaggaagaaaaccgcgccgagaagtacaccttctccgcggctggcctccacgtcgacctgtcgaagaatctgcttgacgacgccaccctcaccaagctccttgcactgaccgaagaatctggccttcgcgaacgcattgacgcgatgtttgccggtgaacacctcaacaacaccgaagaccgcgctgtcctccacaccgcgctgcgccttcctgccgaagctgatctgtcagtagatggccaagatgttgctgctgatgtccacgaagttttgggacgcatgcgtgacttcgctactgcgctgcgctcaggcaactggttgggacacaccggccacacgatcaagaagatcgtcaacattggtatcggtggctctgacctcggaccagccatggctacgaaggctctgcgtgcatacgcgaccgctggtatctcagcagaattcgtctccaacgtcgacccagcagacctcgtttctgtgttggaagacctcgatgcagaatccacattgttcgtgatcgcttcgaaaactttcaccacccaggagacgctgtccaacgctcgtgcagctcgtgcttggctggtagagaagctcggtgaagaggctgtcgcgaagcacttcgtcgcagtgtccaccaatgctgaaaaggtcgcagagttcggtatcgacacggacaacatgttcggcttctgggactgggtcggaggtcgttactccgtggactccgcagttggtctttccctcatggcagtgatcggccctcgcgacttcatgcgtttcctcggtggattccacgcgatggatgaacacttccgcaccaccaagttcgaagagaacgttccaatcttgatggctctgctcggtgtctggtactccgatttctatggtgcagaaacccacgctgtcctaccttattccgaggatctcagccgttttgctgcttacctccagcagctgaccatggaatcaaatggcaagtcagtccaccgcgacggctcccctgtttccactggcactggcgaaatttactggggtgagcctggcacaaatggccagcacgctttcttccagctgatccaccagggcactcgccttgttccagctgatttcattggtttcgctcgtccaaagcaggatcttcctgccggtgagcgcaccatgcatgaccttttgatgagcaacttcttcgcacagaccaaggttttggctttcggtaagaacgctgaagagatcgctgcggaaggtgtcgcacctgagctggtcaaccacaaggtcatgccaggtaatcgcccaaccaccaccattttggcggaggaacttaccccttctattctcggtgcgttgatcgctttgtacgaacacatcgtgatggttcagggcgtgatttgggacatcaactccttcgaccaatggggtgttgaactgggcaaacagcaggcaaatgacctcgctccggctgtctctggtgaagaggatgttgactcgggagattcttccactgattcactgattaagtggtaccgcgcaaataggtag

SEQ ID NO: 7 - DNA, 6126 bp, Artificial Sequence
  Synthetic pJET-EX vector
gcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggccttttcatgattacgggcagatcttcgcctttgacgaatgggccgcgagcgaccagcccgacccccgccccgccacctgacaccagccattggggaggccgccatgcaaggcggcctccctgcgggaaccctgcgtcatggacaccatgctcacgacccagaccatcctctctctcctgcccgcccggtatgccgcggatgcggttgtcatcttctccttcctcatttccggctgtgcgctcgtcgcgcgcttctggcggccacccgcagccgggtcgaaatgggtggtcgtgtggacctttgtaaccgccatggcgcaactgcgtggctggagcaggccccctgacaggaaaggcgatgccacggataagaaaccgtaaagaggtttcgggtgaagcttttttttaaaagattctgaagaaaactgcctttttaacaaacagcagggcaaaaatgatgctgcgtaaacttggctgccgccctgccgaaaggcgtgcgcgccagcccatgctcacaaccatgcggggcttcatggcccgccgcgcgccacagcacctgaaccgcgatggcatcgatcccgccccgctcatgctgggcaatgatgtgctgggtgactgcacggcggcgggcataggcaaccatatccgcgccactgccgcacttgcgggctatcaggtggcgatggatacgcccgatgccgtgcggttctacgcgctttccaccggttatgtgcccggcaacccggccaccgatcatggcggtgtggaagtggatgtgctgagcaggtcgactctagatatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattattatccgctcataattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccgcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagtatagggcgaattcgtagcgcaggaagaaagccaccagcgcccacaggggcagggccatgagcaggctgaaaaagatgccactcgcggcggaataccggcggcgggcagggacagtcactcgctgggcagcaggctgggaaaccgtctgtgtcagggcgataccatcaaacgacatgcgcttagggccttagaaactgaaggaaaggggaaaagcacccccaattgtggagtagcaccacaatcctgccttaaaaataacacgatctgctgtcaatcacttttaattaaactgccatcattatcgctgcctgcatctgcgcagggggctataaaatctggcattaacagacacttccataaaagttacgggttccgcccctgcccggcagcagccagcgcagtatggctttccgtgccatagggtgcggacccgtaccccgaaatgcatctgttcggccacgattcccgcccagcgggcttgtggcctgcaaccggggttccatctgccgcagggccgcgcgctgcgccggggcaatggcccgatcgggtcaagccggtacgcgacggcaggcgtgagaaaaatctgttcgtatcagccagtcctgaaatttcacgggcgggcgcatgctttcttttgctgcctgcatgggcgcgccctatatttcatcttgtcaggagcgaaaagacaacgcgattaccctgaccgcgaaagtataatggcataattcatgcattatacagaacagatacctgcatataaatagatcagggctgtcatcatgccctgtcgagaggatcagatcggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacatagatctcccgggtaccgagctctctagaaagaaggagggacgagctattgatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacagggacgagctattgattgggtaccgagctcgaattcgtacccggggatcctcta

SEQ ID NO: 8 - DNA, 39 bp, Artificial Sequence
  Synthetic primer
atgcctgcaggtcgactagcgcaggaagaaagccaccag

SEQ ID NO: 9 - DNA, 39 bp, Artificial Sequence
  Synthetic primer
agttggatcctctagagatctgatcctctcgacagggca

SEQ ID NO: 10 - DNA, 40 bp, Artificial Sequence
  Synthetic primer
taccgagctcgaattccatgattacgggcagatcttcgcc

SEQ ID NO: 11 - DNA, 39 bp, Artificial Sequence
  Synthetic primer
gacggccagtgaattctcagcacatccacttccacaccg

SEQ ID NO: 12 - DNA, 40 bp, Artificial Sequence
  Synthetic primer
cccggggatcctctaatgaaaaacatcaatccaacgcaga

SEQ ID NO: 13 - DNA, 38 bp, Artificial Sequence
  Synthetic primer
agcttgcatgcctgcttaaccgcgccacgctttatagc

SEQ ID NO: 14 - DNA, 38 bp, Artificial Sequence
  Synthetic primer
cccggggatcctctaatggcggacatttcgaccaccca

SEQ ID NO: 15 - DNA, 38 bp, Artificial Sequence
  Synthetic primer
agcttgcatgcctgcctacctatttgcgcggtaccact

SEQ ID NO: 16 - DNA, 40 bp, Artificial Sequence
  Synthetic primer
cccggggatcctctaatgccgtcaagtacgatcaataaca

SEQ ID NO: 17 - DNA, 38 bp, Artificial Sequence
  Synthetic primer
agcttgcatgcctgcttaagcttcaacctcggagcggt

SEQ ID NO: 18 - DNA, 23 bp, Artificial Sequence
  Synthetic primer
tagcgcaggaagaaagccaccag

SEQ ID NO: 19 - DNA, 23 bp, Artificial Sequence
  Synthetic primer
tcagcacatccacttccacaccg

SEQ ID NO: 20 - protein, 320 aa, Escherichia coli
MIKKIGVLTSGGDAPGMNAAIRGVVRSALTEGLEVMGIYDGYLGLYEDRMVQLDRYSVSDMINRGGTFLGSARFPEFRDENIRAVAIENLKKRGIDALVVIGGDGSYMGAMRLTEMGFPCIGLPGTIDNDIKGTDYTIGFFTALSTVVEAIDRLRDTSSSHQRISVVEVMGRYCGDLTLAAAIAGGCEFVVVPEVEFSREDLVNEIKAGIAKGKKHAIVAITEHMCDVDELAHFIEKETGRETRATVLGHIQRGGSPVPYDRILASRMGAYAIDLLLAGYGGRCVGIQNEQLVHHDIIDAIENMKRPFKGDWLDCAKKLY

SEQ ID NO: 21 - DNA, 963 bp, Escherichia coli
atgattaagaaaatcggtgtgttgacaagcggcggtgatgcgccaggcatgaacgccgcaattcgcggggttgttcgttctgcgctgacagaaggtctggaagtaatgggtatttatgacggctatctgggtctgtatgaagaccgtatggtacagctagaccgttacagcgtgtctgacatgatcaaccgtggcggtacgttcctcggttctgcgcgtttcccggaattccgcgacgagaacatccgcgccgtggctatcgaaaacctgaaaaaacgtggtatcgacgcgctggtggttatcggcggtgacggttcctacatgggtgcaatgcgtctgaccgaaatgggcttcccgtgcatcggtctgccgggcactatcgacaacgacatcaaaggcactgactacactatcggtttcttcactgcgctgagcaccgttgtagaagcgatcgaccgtctgcgtgacacctcttcttctcaccagcgtatttccgtggtggaagtgatgggccgttattgtggagatctgacgttggctgcggccattgccggtggctgtgaattcgttgtggttccggaagttgaattcagccgtgaagacctggtaaacgaaatcaaagcgggtatcgcgaaaggtaaaaaacacgcgatcgtggcgattaccgaacatatgtgtgatgttgacgaactggcgcatttcatcgagaaagaaaccggtcgtgaaacccgcgcaactgtgctgggccacatccagcgcggtggttctccggtgccttacgaccgtattctggcttcccgtatgggcgcttacgctatcgatctgctgctggcaggttacggcggtcgttgtgtaggtatccagaacgaacagctggttcaccacgacatcatcgacgctatcgaaaacatgaagcgtccgttcaaaggtgactggctggactgcgcgaaaaaactgtattaa

SEQ ID NO: 22 - DNA, 3576 bp, Artificial Sequence
  Synthetic pTSa-EX1 vector
gaattcagccagcaagacagcgatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatcacctgtaagtcggacgttccgatcacctgtaacgatgcgtccggcgtagaggatccggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacagggacgagctattgattgggtaccgagctcgaattcgtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcaagaacatgtgagcacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagcatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatttttaaggcagttattggtgcccttaaacgcctggttgctacgcctgaataagtgataataagcggatgaatggcagaaattc

SEQ ID NO: 23 - DNA, 33 bp, Artificial Sequence
  Synthetic primer
cggcgtagaggatcaggagcttatcgactgcac

SEQ ID NO: 24 - DNA, 28 bp, Artificial Sequence
  Synthetic primer
ccggcgtagagaatccacaggacgggtg

SEQ ID NO: 25 - DNA, 27 bp, Artificial Sequence
  Synthetic primer
ctgtggattctctacgccggacgcatc

SEQ ID NO: 26 - DNA, 29 bp, Artificial Sequence
  Synthetic primer
aagggcatcggtcgtcgctctcccttatg

SEQ ID NO: 27 - DNA, 39 bp, Artificial Sequence
  Synthetic primer
cgtacccggggatccatgattaagaaaatcggtgtgttg

SEQ ID NO: 28 - DNA, 39 bp, Artificial Sequence
  Synthetic primer
gactctagaggatccttaatacagttttttcgcgcagtc

SEQ ID NO: 29 - DNA, 40 bp, Artificial Sequence
  Synthetic primer
cttgatatcgaattcttctcatgtttgacagcttatcatc

SEQ ID NO: 30 - DNA, 36 bp, Artificial Sequence
  Synthetic primer
gggctgcaggaattcgaatttctgccattcatccgc

SEQ ID NO: 31 - DNA, 37 bp, Artificial Sequence
  Synthetic primer
cttgatatcgaattaggcctgtcatcgtctatatacg

SEQ ID NO: 32 - DNA, 42 bp, Artificial Sequence
  Synthetic primer
cgtgttgttcgaattcgatggatattcctccagtatcatgtg

SEQ ID NO: 33 - DNA, 32 bp, Artificial Sequence
  Synthetic primer
catcgaattcgaacaacacgccgatgtatgac

SEQ ID NO: 34 - DNA, 40 bp, Artificial Sequence
  Synthetic primer
acatgagaagaattgacagatccggtcagttcacattatc

SEQ ID NO: 35 - DNA, 39 bp, Artificial Sequence
  Synthetic primer
cagaaattcgaattgcgatcatcaccaaccaggaaattc

SEQ ID NO: 36 - DNA, 37 bp, Artificial Sequence
  Synthetic primer
gggctgcaggaattgggtatttcaggcggcagtaaag

SEQ ID NO: 37 - DNA, 40 bp, Artificial Sequence; identical to SEQ ID NO: 29
  Synthetic primer

SEQ ID NO: 38 - DNA, 36 bp, Artificial Sequence; identical to SEQ ID NO: 30
  Synthetic primer

SEQ ID NO: 39 - DNA, 4381 bp, Artificial Sequence
  Synthetic pMKO vector
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccaacttcggcggcgcccgagcgtgaacagcacgggctgaccaacctgtgcgcgcgcggcggctacgtcctggcggaagccgaagggacgcggcaggtcacgctggtcgccacggggcacgaggcgatactggcgctggcggcacgcaaactgttgaaggacgcaggggttgcggcggctgtcgtatcccttccatgctgggaactgttcgccgcgcaaaaaatgacgtatcgtgccgccgtgctgggaacggcaccccggatcggcattgaagccgcgtcagggtttggatgggaacgctggcttgggacagacgggctgtttgttggcattgacgggttcgggacggccgccccggaccagccggacagcgcgactgacatcacgccggaacggatctgccgcgacgcgctgcgtctggtccgtcccctgtccgataccctgactgaaccggcgggaggaaacggcgcgccgcccgggatgacatcggccgatgtcagtgtgtgaaatgtcagaccttacggagaaaataagaaaagatctcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 40 - DNA, 2974 bp, Artificial Sequence
  Synthetic pJET1.2 vector
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagatatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattattatccgctcataattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 41 - DNA, 35 bp, Artificial Sequence
  Synthetic primer
ggatcagatctggctgtgcaggtcgtaaatcactg

SEQ ID NO: 42 - DNA, 39 bp, Artificial Sequence
  Synthetic primer
agttggatcctctagaaaaaggccatccgtcaggatggc